(19) United States
(12) Patent Application Publication
(10) Pub. No.: US 2009/0304764 A1
(43) Pub. Date: Dec. 10, 2009

(54) Bone Regeneration Element for Stabilising Artificial Tooth Roots

(76) Inventor: Ulrich Breckwoldt, Neustadt in Holstein (DE)

Correspondence Address: Clements Bernard PLLC, 1901 Roxborough Road, Suite 250, Charlotte, NC, 28211, US

(21) Appl. No.: 11/921,388

(22) PCT Filed: Jun. 1, 2006
(86) PCT No.: PCT/EP2006/062844

§ 371 Date: Jan. 11, 2008

(30) Foreign Application Priority Data
Jun. 1, 2005 (DE) 10 2005 024 978.7

(51) Int. Cl. A61F 2/00

(52) U.S. Cl. 424/423

(57) ABSTRACT

A bone regeneration element for stabilizing artificial tooth roots (10) in a bone (12) comprises a ring-segment-shaped or full-ring-shaped annular body (20,22) which at least partially surrounds the artificial tooth root (10). The ring-segment-shaped or full-ring-shaped annular body (20,22) is produced at least partially from a porous material. A filling space (30) formed between the ring-segment-shaped or full-ring-shaped annular body (20,22) and the artificial tooth root (10) is filled with bone dust or bone chips and the patient's own blood. As a result of the porosity of the material, vessels can grow therein and the artificial tooth root can thus be fixed.

The invention relates to a bone regeneration element for tooth implantation.

In dental implantology, artificial tooth roots which usually are made of titanium or the like, are fixed in the jawbone, e.g. by screwing them into place. The artificial root of the tooth and the dental implant, respectively, will then support the artificial structure such as e.g. the artificial crown of the tooth or the replacement tooth. For cases where the quantity of the existing bone substance is insufficient for fixing the artificial root, it is known to effect a corresponding osteogenisis e.g. by implantation of autologous bone which is taken from the hip bone, for instance. Such an approach involves a difficult and time-consuming surgical intervention e.g. on the hip. A procedure of this type cannot be performed directly in a conventionally equipped dentist's office.

It is an object of the invention to provide a bone regeneration element which is suited to enhance a controlled osteogenisis directly around the artificial root of the tooth and around the implant, respectively.

According to the invention, the above object is achieved by the features indicated in claim 1.

The bone regeneration element of the invention serves for the formation of bones which subsequently will be used to hold an artificial root or an implant. For this purpose, the bone regeneration element comprises an annular body shaped as a ring segment or an annular body shaped as a full ring. This ring-segment-shaped or full-ring-shaped body is at least partially made of a porous material. For osteogenisis of the artificial root, the bone regeneration element of the invention is arranged on the jawbone in such a manner that the element will at least partially surround the artificial root. To accomplish, with the aid of the bone regeneration element, a long-term stabilization of the artificial root or the implant by use of newly formed bones, a gap or filling space existing between the bone regeneration element and the artificial root will be filled with filling material. Depending on its nature and dimension, the bone regeneration element alone or in combination with the filling material is already effective to improve a primary stability which will enhance an osseointegration of the artificial root and respectively the tooth implant as well as the new formation of bone around the artificial root, and which further will establish the precondition for the durable secondary stability.

The filling material is a synthetic or natural bone replacement material; this material can be biologically inert or bioabsorbable. Suitable as synthetic filling materials are e.g. calcium phosphates such as hydroxylapatite, tricalcium phosphate, bioactive glasses and/or calcium sulfates. Also mixtures of these materials, such as e.g. biphasic calcium phosphates, can be of particular usefulness. A natural filling material can be of human or xenogeneic origin; particularly it can be an autologous bone transplant which is obtained extra-orally or intra-orally. Further, use can be made of bone materials of other origins which are suitable for thus application. Autologous bone materials can be prepared of bone chips or be available in ground form or as a bone block. Suitable as natural filling materials are e.g. bone materials from the chin, from the retromolar jaw region, from the upper jaw, from the nasal thorn, from the iliac crest and from the skull cap as well as bovine materials, algae or corals as well as collagens. All of these materials can also be combined with each other in a suitable manner and, if required, will be mixed with liquids such as e.g. the patient's own blood or bone marrow. Preferably, use is made of a mixture of bone chips or bonemeal and the patient's own blood. It is a common feature of the above mentioned filling materials that, because of their composition, they are effective to enhance the regeneration of new bone structures.

Due to the porosity of at least a part of the ring-segment-shaped or full-ring-shaped annular body and due to the provision of suitable Filling material which preferably consists of endogenous substances, osteogenisis around the artificial tooth root and the tooth implant, respectively, is enhanced, while vessels can sprout into the relevant region through the porous material. This sprouting of vessels is the precondition for the generation of bones and respectively the integration of the ring-segment-shaped or full-ring-shaped annular body into the newly formed bone. As a result, the ring-segment-shaped or full-ring-shaped annular body will be integrated into the newly formed bone and thus will be stabilized. Accordingly, in the process, the artificial tooth root and the tooth implant, respectively, is integrated into the newly generating bone (osseointegration).

Conventionally, in the bone formation process for implanting an artificial tooth root, use is made of a filler material which partially consists of autologous bone material. In case of an implanting in the posterior maxilla, there may be used, e.g. for the so-called sinus floor elevation, a very large augmentation volume and thus a large quantity of filling material. In the presently described method, by contrast, a much lower volume of filling material is required since only the region between the ring-segment-shaped or full-ring-shaped body and the artificial tooth root has to be filled with filling material. Thus, according to the invention, the transplant quantity of autologous bone material can be considerably reduced, thus rendering it possible to abstain more frequently from extra-oral bone generation.

By way of a further example, the ring-segment-shaped annular body can be used primarily in the anterior region both of the maxilla and of the mandible for bone regeneration in case of loss of the vestibular bone lamella. In doing so, the ring-segment-shaped body serves for reducing the required quantity of filling material and for stabilizing the filling material around the exposed tooth implant which had been safely anchored by its apical region in the residual bone. This application makes it possible to reconstruct an aesthetic jaw ridge profile.

Further, the inventive intervention can be performed by way of a mere out-patient treatment in any conventionally equipped dentist's office; this is made possible because in intra-oral bone generation, only local anesthesia is needed and in most cases an extra-oral generation of bone material can be omitted.

The bone regeneration element of the invention offers the particular advantage that the surrounding area of the tooth implant can be exactly defined. Particularly, the intermediate or filling space is limited and the filling quantity is thus clearly defined. Especially, the filling quantity is noticeably reduced as compared to conventional methods.

Preferably, the inventive annular body comprises a body shaped as a ring segment or a body shaped as a full ring. This body will thus be a tubular body or a tube-segment-shaped body formed in the manner of a hollow cylinder or a hollow-cylinder segment. In such a configuration, the annular body is preferably circular or is formed as a segment of a circle but can also have a polygonal shape. Particularly, the outer contour of the ring-segment-shaped or full-ring-shaped body can have a shape which has been adapted or is adaptable to the given conditions. For instance, an adapting process can be performed on the ring-segment-shaped or full-ring-shaped body on the basis of the residual natural bone still existing in the immediate vicinity.

Preferably, however, the ring-segment-shaped or full-ring-shaped body is a circular body or a body formed as a partial circle.

To effect a reliable sprouting of the vessels, the annular body is, at least within ring segments, made of a porous material throughout the wall thickness. The porous material thus extends from an outer wall all the way to an inner wall of the annular body, thus allowing a sprouting of the vessels from the outside to the inside into the filling material. Particularly in case of ring-segment bodies, it is not required for the porous material to extend through the whole wall thickness, since a sprouting of vessels is possible also via the lateral opening of the ring-segment-shaped body. The porosity of the material will in this case serve particularly for stabilizing the ring-segment-shaped body and or the artificial tooth root and respectively the tooth implant by sprouting of vessels or by new formation of bones. This applies particularly to bodies shaped as full rings since the vessels will then be allowed to grow also the axial direction from the jaw bones into the filling material. Also with regard to this process, the porosity of the material serves for fixing the annular body and of the artificial tooth root and respectively the tooth implant by the sprouting of vessels into the porous material, and for a new formation of bones.

The mechanical strength of the full ring-shaped or ring-segment-shaped body is influenced above all by the composition of the body. What is important is a certain degree of porosity; good results are obtained by an overall porosity of 20% to 80%. The range from 30% to 70% is preferred, while the range from 40% to 60% is especially preferred. The pores are interconnected throughout the cross section of the body in order to guarantee an open connection for inward-growing cells and diffusing nutrients, gases and liquids between the inner and outer sides of the body.

To still further improve the good connection between the forming new bone material and the annular body, an inner wall and/or an outer wall of the annular body have a high roughness. This is effective to improve the osteoconductivity.

Further, particularly the surface of the inner wall and, if required, also of the outer wall is enlarged. Also in this manner, the firm hold of the annular body in the newly generated bone is improved by increased osseointegration.

If the annular body is shaped as a full ring, the inner diameter has to be adapted to the outer diameter of the artificial tooth root in such a manner that the intermediate space is sufficiently wide for insertion of a filling material into it, a preferred width being in the range of 1-2 mm. The wall thickness and the height of the full-ring-shaped body are selected such that the osseointegration of the artificial tooth root and of the tooth implant, respectively, is made possible. The wall thickness is preferably selected in the range of 1-2 mm and/or the height is preferably selected in the range of 3-7 mm. A ring-segment-shaped body will preferably have corresponding dimensions.

With particular preference, the ring-segment-shaped or full-ring-shaped annular body is provided with a bearing element so that a plurality of annular bodies, particularly two annular bodies, can be arranged in a simple manner with exact positional accuracy relative to each other. Thus, for instance, two annular bodies can be arranged onto each other with exact positional accuracy. Position-defining elements can comprise e.g. a projection formed on one annular body that is configured for form-locking engagement with a recess formed on a second annular body. Also, a preferably annular groove can be provided to be engaged by a likewise annular spring of the second annular body. It is particularly preferred if the position-defining elements are arranged on mutually confronting end faces of the ring bodies.

Particularly for fixation of ring bodies on massively receded bones, the annular body is preferably connected to a holding element. The holding element is e.g. a clamping member such as a clamping screw or a clamping nut, for instance. In cases where the still available natural bone structure is very small, a bone regeneration element fitted with a holding or clamping element can establish the required primary stability of the artificial tooth root. In the present embodiment, the lower annular element comprises a screw thread and/or has a large outer diameter. The screw thread or a clamping element are effective to lend the bone regeneration element a firm seat on the artificial tooth root. A larger diameter of the lower annular element will increase the abutment area and thus the positional stability of the artificial tooth root in the residual bone. A bone regeneration element of the above design is distinguished—apart from the above advantages—by an improved primary stability of the overall system encompassing the bone regeneration element, the artificial tooth root, the filling material and the natural residual bone.

As a material for producing the ring-segment-shaped or full-ring-shaped body which is preferably made of a porous material, there is suited e.g. biologically inert material such as titanium, for instance. Further, use can be made of biodegradable materials such as e.g. polylactide/glycolide mixtures, polyanhydrides, chitosan reinforced by calcium phosphate cements, or calcium phosphates such as e.g. hydroxylapatite or tricalcium phosphate as a base material. Further still, concerning the materials in question, reference may be made to the base materials which were mentioned for the filling material. Like the filling material, also the material of the ring-segment-shaped or full-ring-shaped body should be capable of enhancing the new formation of bone structures. Due to the shaping function or container function of the ring-segment-shaped or full-ring-shaped body, the use of freshly removed natural bone material is less eligible. In order to nonetheless be able to enhance a new formation of bones in the region of the ring-segment-shaped or full-ring-shaped body, a combination of the material of the body with osteoinductive substances can be of particular advantage. Examples of such substances are growth-stimulating factors, hormones or adhesive molecules.

Bone regeneration elements complemented in this manner are effective, apart from the stabilizing function, to function as a bone growth matrix. In the process, the osteoinductive substances mentioned by way of example can be released or remain tightly connected to the bone regeneration element.

Since the filling material preferably comprises bone meal or bone chips mixed with the patient's own blood and since, because of the inventive provision of the ring-segment-shaped or full-ring-shaped body, only small quantities of filling materials are required, the bone chips obtained during the drilling of the residual bone and thus being available for the insertion of a part of the artificial tooth root, will already be sufficient. Should the quantity of bone meal or bone chips obtained in the process not suffice, bone material can be obtained from adjacent regions by scraping or the like, i.e. without the necessity of a further intervention.

The invention also relates to a bone regeneration system comprising an artificial tooth root made e.g. of titanium, as well as at least one bone regeneration element as described above. Preferably, the bone regeneration element additionally comprises a filling material to be introduced into the filling space. This filling material can be mixed or enriched with blood and bone meal/chips of the patient.

A preferred embodiment of the invention will be explained in greater detail hereunder with reference to the drawings.

In the drawings—

FIG. 1 is a schematic side view of an artificial tooth root inserted in a bone and surrounded by bone regeneration elements according to the invention,

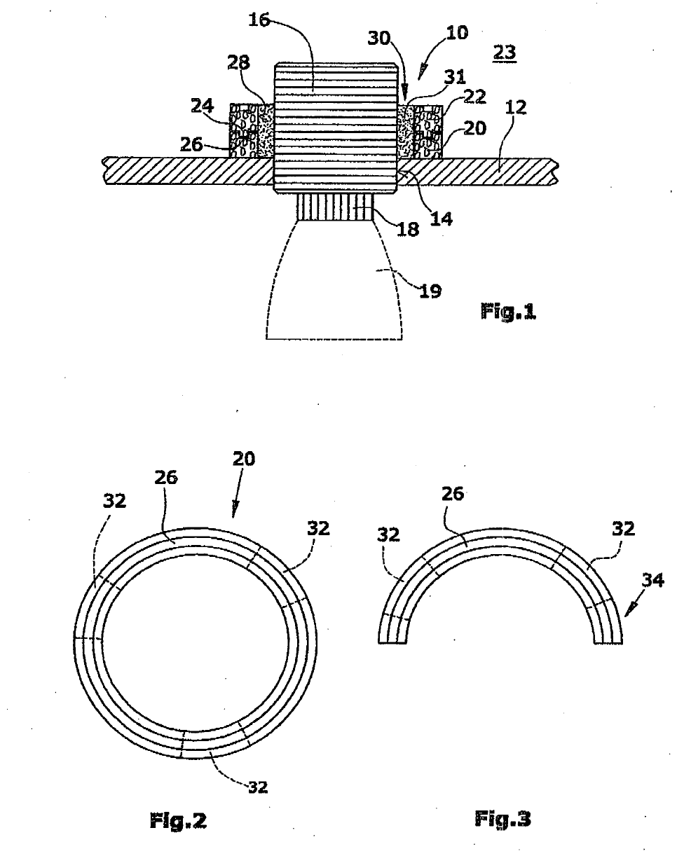

FIG. 2 is a schematic plan view of a body shaped as a full ring,

FIG. 3 is a schematic plan view of a body shaped as a ring segment,

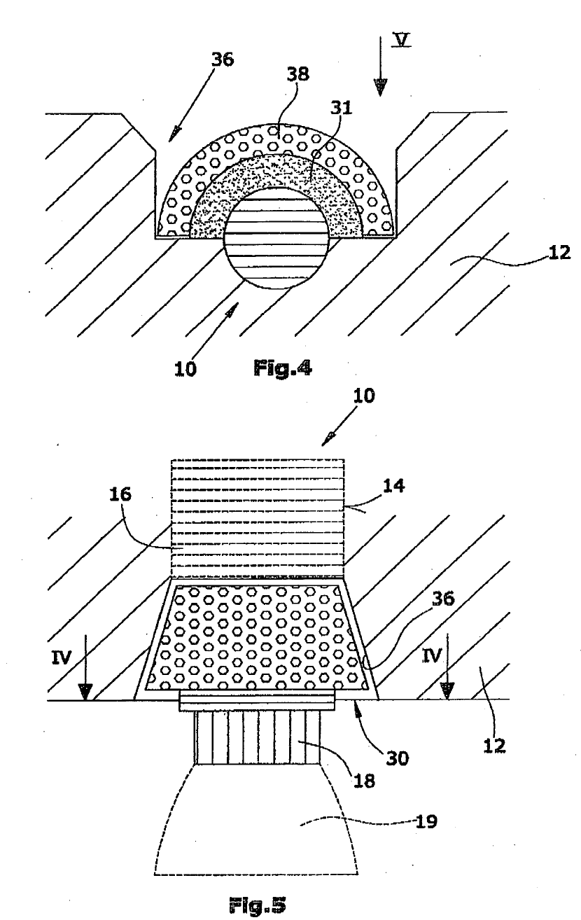

FIG. 4 is a schematic sectional view of an artificial tooth root inserted into a bone, with the root partially surrounded by filling material stabilized with the aid of a ring-segment-shaped bone regeneration element, and

FIG. 5 a schematic plan view of the artificial tooth root shown in FIG. 4 together with a bone regeneration element and filling material as seen in the direction indicated by arrow V in FIG. 4.

For stabilizing an artificial tooth root 10, a bore 14 will be generated in a residual bone 12 (FIG. 1), and a cylindrical portion 16 of the tooth root will be screwed into this bore. Subsequently, the dental prosthesis is screwed or fastened to a likewise cylindrical portion 18 which preferably has a smaller diameter and is part of the artificial tooth root or the tooth implant or is connected to the tooth or implant.

In the illustrated exemplary embodiment, to apply the filling material 31 to the artificial tooth root 10, two bodies 20,22 shaped as full rings will be positioned to surround the region 16. In the exemplary embodiment shown, the upper full-ring-shaped body 22 comprises a projection formed as a ring segment and pointing in the direction of the other full-ring-shaped body 20, said projection engaging an opposite groove 26 of the lower full-ring-shaped body 20. Both said projection 24 and the groove 26 are arranged on mutually opposite end sides of the full-ring-shaped bodies. In this manner, the position of the two full-ring-shaped bodies 22,20 relative to each other is defined.

The full ring bodies 20,22 are arranged within the maxillary sinus 23.

Between the inner walls 28 of the ring bodies 20,22 and the region 16 of the artificial tooth root 10, a filling space 30 is formed. This filling space 30 will be filled with filling material 31 comprising preferably bone meal or bone chips as well as blood of the patient. Since the full ring bodies 20,22 preferably completely consist of porous material, tissue will sprout through the full ring bodies into the filling space 30, thus effecting a good fixation of the full ring bodies 20,22 as well as the artificial tooth root in the bone 12.

To make it possible, in the extremely thin residual bone 12, to obtain a certain degree of stability already before the formation of the new bone tissue, the regeneration element—in the illustrated example the regeneration element 20—which is provided adjacent to bone 12 can be fixedly connected to the tooth implant 10. In this case, the relevant region will be left without a filling space 30, preferably completely. The connection can be realized by clamping or by threaded engagement.

The annular bodies 20,22 can be bodies shaped as full rings (FIG. 2) but also bodies 34 shaped as ring segments (FIG. 3).

The ring-segment-shaped or full-ring-shaped body is preferably completely made of porous material. However, it can also be provided that only partial regions, particularly annular segments 32, consist of porous material.

In reference to a further preferred embodiment of the invention (FIGS. 4 and 5), identical or similar components will be identified by the same reference numerals.

This exemplary embodiment relates to the arrangement of a tooth implant 10 in a bone which is not—as described in connection with FIG. 1—too thin but is too narrow or includes a recess 36. Projecting from bone 36 is a part of the tooth implant. The tooth implant is surrounded by a ring-segment-shaped bone regeneration element 38 which corresponding to the above described regeneration elements is made of a porous material or includes porous material. The shape of the regeneration element 38 is adapted to the recess 36. Such recesses will generally have the shape illustrated in FIG. 5. With the aid of normally just slight adaptations performed on the recess 36, it is rendered possible to use standardized regeneration elements 38. In the illustrated exemplary embodiment, the regeneration element 38 is a ring segment of a part-frustoconical shape. The insertion of filling material 31 into the filling space 30 is performed as described in the context of the exemplary embodiment according to FIG. 1.

1. A bone regeneration element for stabilizing artificial tooth roots (10) in a bone (12), said bone regeneration element comprising
a ring-segment-shaped or full-ring-shaped annular body (20,22,34,38) at least partially surrounding the artificial tooth root (10),
said ring-segment-shaped or full-ring-shaped annular body (20,22,34,38) at least partially comprising porous material.

2. The bone regeneration element according to claim 1, characterized in that, in partial regions and particularly within ring segments (32,38), said annular body comprises a porous material throughout the wall thickness.

3. The bone regeneration element according to claim 1, characterized in that said porosity is higher than 30%, particularly higher than 50%.

4. The bone regeneration element according to claim 1, characterized in that the inner wall (28) of said annular body has a high roughness.

5. The bone regeneration element according to claim 1, characterized in that the inner wall (28) of said annular body (20,22,34,38) comprises an enlarged surface.

6. The bone regeneration element according to claim 1, characterized in that, between the inner wall (28) of said annular body (20,22,34,38) and the artificial tooth root (10), a filling space (30) is formed to be filled with filling material.

7. The bone regeneration element according to claim 1, characterized in that the inner diameter of said ring-segment-shaped or full-ring-shaped annular body (20,22,34,38) is 5-7 mm and/or the wall thickness is 1-2 mm and/or the heights is 3-7 mm.

8. The bone regeneration element according to claim 1, characterized in that said annular body (20,22,34,38) is provided with a position-defining element (24,26) for precise positioning of said annular body relative to a second annular body (20,22,34,38).

9. The bone regeneration element according to claim 1, characterized by a holding element connected to said annular body (20,22,34,38) and provided for fixation to a bone (12).

10. The bone regeneration element according to claim 1, characterized by a fastening element connected to the bone regeneration element and provided for attachment to an artificial tooth root (10), particularly by clamping.

11. A bone regeneration system, comprising
an artificial tooth root (10), and
at least one bone regeneration element according to claim 1.

12. The bone regeneration system according to claim 11, comprising a filling material adapted to be inserted into a filling space (30) between the inner wall (28) of said annular body (20,22,34,38) and the artificial tooth root (10).

13. The bone regeneration system according to claim 12, characterized in that said filling space (30) is formed by a distance between the inner wall (28) of said annular body (20,22,34,38) and the artificial tooth root (10), said distance preferably being 1 to 2 mm.

14. The bone regeneration system according to claim 1, characterized by the use of a plurality of bone regeneration elements which are preferably connected to each other by position-defining elements.

15. The bone regeneration system according to claim 14, characterized in that one of said bone regeneration elements is directly connected to the tooth root (10) and is formed as a clamping element.
